(19) United States
(12) Patent Application Publication
(10) Pub. No.: US 2026/0301914 A1
(43) Pub. Date: Oct. 1, 2026

(54) PHYSICIAN ESCALATION STRATEGY FOR ONLINE ADAPTIVE RADIOTHERAPY

(71) Applicant: Elekta LTD., Montreal (CA)

(72) Inventors: Martin Emile Lachaine, Montreal (CA); Tony Falco, La Prairie (CA)

(21) Appl. No.: 19/483,406

(22) PCT Filed: Apr. 25, 2024
(86) PCT No.: PCT/CA2024/050545

§ 371 Date: Nov. 11, 2025

(30) Foreign Application Priority Data
May 11, 2023 (GB) 2307023.8

(51) Int. Cl. G16H 20/40; A61N 5/10; G16H 30/40

(52) U.S. Cl. CPC G16H 20/40; A61N 5/107; G16H 30/40

(57) ABSTRACT

Systems and techniques may be used for determining a workflow path using image-guided radiation therapy or adaptive radiotherapy. An example technique may include receiving a planning image of a patient, and performing a determination based on the planning image, using a first model, whether to proceed with an adaptive workflow path or an image-guided radiation therapy (IGRT) path for radiation treatment of the patient. In response to determining to proceed with the adaptive workflow path, the example technique may include automatically generating a contour of the image, and performing a determination based on the contour, using a second model, whether to proceed with the adaptive workflow path or the IGRT path for the radiation treatment of the patient. An indication of the determination may be output based on the contour.

### BACKGROUND

Radiation therapy or “radiotherapy” may be used to treat cancers or other ailments in mammalian (e.g., human and animal) tissue. One such radiotherapy technique is referred to as “gamma knife,” by which a patient is irradiated using a number of lower-intensity gamma rays that converge with higher intensity and high precision at a targeted region (e.g., a tumor). In another example, radiotherapy is provided using a linear accelerator (“linac”), whereby a targeted region is irradiated by high-energy particles (e.g., electrons, high-energy photons, and the like). In another example, radiotherapy is provided using a heavy charged particle accelerator (e.g. protons, carbon ions, and the like), The placement and dose of the radiation beam is accurately controlled to provide a prescribed dose of radiation to the targeted region. The radiation beam is also generally controlled to reduce or minimize damage to surrounding healthy tissue, such as may be referred to as “organ(s) at risk” (OARs). Radiation may be referred to as “prescribed” because generally a physician orders a predefined dose of radiation to be delivered to a targeted region such as a tumor.

Generally, ionizing radiation in the form of a collimated beam is directed from an external radiation source toward a patient. Modulation of a radiation beam may be provided by one or more attenuators or collimators (e.g., a multi-leaf collimator). The intensity and shape of the radiation beam may be adjusted by collimation avoid damaging healthy tissue (e.g., OARs) adjacent to the targeted tissue by conforming the projected beam to a profile of the targeted tissue.

The treatment planning procedure may include using a three-dimensional image of the patient to identify the target region (e.g., the tumor) and such as to identify critical organs near the tumor. Creation of a treatment plan may be a time consuming process where a planner tries to comply with various treatment objectives or constraints (e.g., dose volume histogram (DVH) objectives or other constraints), such as taking into account importance (e.g., weighting) of respective constraints in order to produce a treatment plan that is clinically acceptable. This task may be a time-consuming trial-and-error process that is complicated by the various organs at risk (OARs) because as the number of OARs increases (e.g., about thirteen for a head-and-neck treatment), so does the complexity of the process. OARs distant from a tumor may be more easily spared from radiation, but OARs close to or overlapping a target tumor may be more difficult to spare from radiation exposure during treatment.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example but not by way of limitation, various embodiments discussed in the present document.

FIG. 1 illustrates an exemplary radiotherapy system adapted for performing image patient state estimation processing.

FIG. 2 illustrates an exemplary image-guided radiotherapy device.

FIG. 3 illustrates a partially cut-away view of an exemplary system including a combined radiation therapy system and an imaging system, such as a nuclear magnetic resonance (MR) imaging system.

FIG. 4 illustrates an exemplary flow diagram for planning radiotherapy treatment using an IGRT and adaptive radiotherapy workflow.

FIG. 5 illustrates an exemplary user interface showing example rigid and deformable structures.

FIG. 6 illustrates an exemplary diagram showing adaptive radiotherapy configurations.

FIG. 7 illustrates an exemplary block diagram for an adaptive radiotherapy workflow.

FIG. 8 a flowchart of exemplary operations for planning radiotherapy treatment using an IGRT and adaptive radiotherapy workflow.

### DETAILED DESCRIPTION

In the following detailed description, reference is made to the accompanying drawings which form a part hereof, and which is shown by way of illustration-specific embodiments in which the present invention may be practiced. These embodiments, which are also referred to herein as “examples,” are described in sufficient detail to enable those skilled in the art to practice the invention, and it is to be understood that the embodiments may be combined, or that other embodiments may be utilized and that structural, logical and electrical changes may be made without departing from the scope of the present invention.

The following detailed description is, therefore, not be taken in a limiting sense, and the scope of the present invention is defined by the appended claims and their equivalents.

Image guided radiation therapy (IGRT) may use computed tomography (CT) imaging, cone beam CT (CBCT), magnetic resonance (MR) imaging, positron-emission tomography (PET) imaging, or the like to obtain a 3D or 4D image of a patient prior to irradiation. For example, a CBCT-enabled linac (linear accelerator) may consist of a kV source/detector affixed to the gantry at a 90 degree angle to a radiation beam, or a MR-Linac device may consist of a linac integrated directly with an MR scanner.

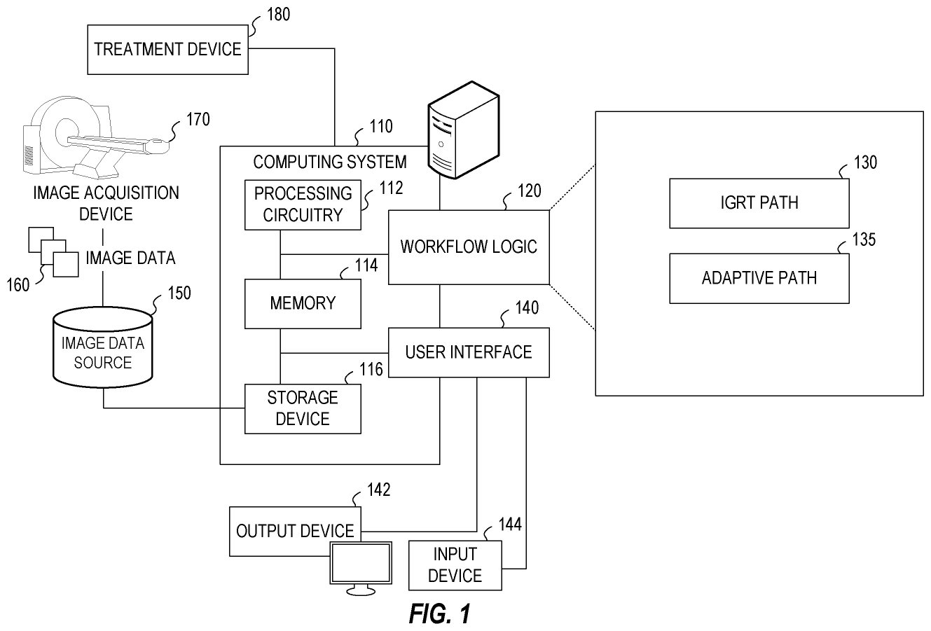

FIG. 1 illustrates an exemplary radiotherapy system adapted for using deep neural networks for real-time motion monitoring. The real-time motion monitoring may be used to determine a patient state to enable the radiotherapy system to provide radiation therapy to a patient based on specific aspects of captured medical imaging data. The radiotherapy system includes an image processing computing system 110 which hosts workflow logic 120. The image processing computing system 110 may be connected to a network (not shown), and such network may be connected to the Internet. For instance, a network can connect the image processing computing system 110 with one or more medical information sources (e.g., a radiology information system (RIS), a medical record system (e.g., an electronic medical record (EMR) / electronic health record (EHR) system), an oncology information system (OIS)), one or more image data sources 150, an image acquisition device 170, and a treatment device 180 (e.g., a radiation therapy device).

The image processing computing system 110 may include processing circuitry 112, memory 114, a storage device 116, and other hardware and software-operable features such as a user interface 140, communication interface, and the like. The storage device 116 may store computer-executable instructions, such as an operating system, radiation therapy treatment plans (e.g., original treatment plans, adapted treatment plans, or the like), software programs (e.g., radiotherapy treatment plan software, artificial intelligence implementations such as deep learning models, machine learning models, and neural networks, etc.), and any other computer-executable instructions to be executed by the processing circuitry 112.

In an example, the processing circuitry 112 may include a processing device, such as one or more general-purpose processing devices such as a microprocessor, a central processing unit (CPU), a graphics processing unit (GPU), an accelerated processing unit (APU), or the like. More particularly, the processing circuitry 112 may be a complex instruction set computing (CISC) microprocessor, a reduced instruction set computing (RISC) microprocessor, a very long instruction Word (VLIW) microprocessor, a processor implementing other instruction sets, or processors implementing a combination of instruction sets. The processing circuitry 112 may also be implemented by one or more special-purpose processing devices such as an application specific integrated circuit (ASIC), a field programmable gate array (FPGA), a digital signal processor (DSP), a System on a Chip (SoC), or the like. As would be appreciated by those skilled in the art, in some examples, the processing circuitry 112 may be a special-purpose processor, rather than a general-purpose processor. The processing circuitry 112 may include one or more known processing devices, such as a microprocessor from the Pentium™, Core™, Xeon™, or Itanium® family manufactured by Intel™, the Turion™, Athlon™, Sempron™, Opteron™, FX™, Phenom™ family manufactured by AMD™, or any of various processors manufactured by Sun Microsystems. The processing circuitry 112 may also include graphical processing units such as a GPU from the GeForce®, Quadro®, Tesla® family manufactured by Nvidia™, GMA, Iris™ family manufactured by Intel™, or the Radeon™ family manufactured by AMD™.

The processing circuitry 112 may also include accelerated processing units such as the Xeon Phi™ family manufactured by Intel™. The disclosed embodiments are not limited to any type of processor(s) otherwise configured to meet the computing demands of identifying, analyzing, maintaining, generating, and/or providing large amounts of data or manipulating such data to perform the methods disclosed herein. In addition, the term “processor” may include more than one processor, for example, a multi-core design or a plurality of processors each having a multi-core design. The processing circuitry 112 can execute sequences of computer program instructions, stored in memory 114, and accessed from the storage device 116, to perform various operations, processes, methods that will be explained in greater detail below.

The memory 114 may comprise read-only memory (ROM), a phase-change random access memory (PRAM), a static random access memory (SRAM), a flash memory, a random access memory (RAM), a dynamic random access memory (DRAM) such as synchronous DRAM (SDRAM), an electrically erasable programmable read-only memory (EEPROM), a static memory (e.g., flash memory, flash disk, static random access memory) as well as other types of random access memories, a cache, a register, a compact disc read-only memory (CD-ROM), a digital versatile disc (DVD) or other optical storage, a cassette tape, other magnetic storage device, or any other non-transitory medium that may be used to store information including image, data, or computer executable instructions (e.g., stored in any format) capable of being accessed by the processing circuitry 112, or any other type of computer device. For instance, the computer program instructions can be accessed by the processing circuitry 112, read from the ROM, or any other suitable memory location, and loaded into the RAM for execution by the processing circuitry 112.

The storage device 116 may constitute a drive unit that includes a machine-readable medium on which is stored one or more sets of instructions and data structures (e.g., software) embodying or utilized by any one or more of the methodologies or functions described herein. The instructions may also reside, completely or at least partially, within the memory 114 and/or within the processing circuitry 112 during execution thereof by the image processing computing system 110, with the memory 114 and the processing circuitry 112 also constituting machine-readable media.

The memory device 114 or the storage device 116 may constitute a non-transitory computer-readable medium. For example, the memory device 114 or the storage device 116 may store or load instructions for one or more software applications on the computer-readable medium. Software applications stored or loaded with the memory device 114 or the storage device 116 may include, for example, an operating system for common computer systems as well as for software-controlled devices. The image processing computing system 110 may also operate a variety of software programs comprising software code for implementing the workflow logic 120 and the user interface 140. Further, the memory device 114 and the storage device 116 may store or load an entire software application, part of a software application, or code or data that is associated with a software application, which is executable by the processing circuitry 112. In a further example, the memory device 114 or the storage device 116 may store, load, or manipulate one or more radiation therapy treatment plans, imaging data, patient state data, dictionary entries, artificial intelligence model data, labels and mapping data, etc. It is contemplated that software programs may be stored not only on the storage device 116 and the memory 114 but also on a removable computer medium, such as a hard drive, a computer disk, a CD-ROM, a DVD, a HD, a Blu-Ray DVD, USB flash drive, a SD card, a memory stick, or any other suitable medium; such software programs may also be communicated or received over a network.

Although not depicted, the image processing computing system 110 may include a communication interface, network interface card, and communications circuitry. An example communication interface may include, for example, a network adaptor, a cable connector, a serial connector, a USB connector, a parallel connector, a high-speed data transmission adaptor (e.g., such as fiber, USB 3.0, thunderbolt, and the like), a wireless network adaptor (e.g., such as a IEEE 802.11/Wi-Fi adapter), a telecommunication adapter (e.g., to communicate with 3G, 4G/LTE, and 5G, networks and the like), and the like. Such a communication interface may include one or more digital and/or analog communication devices that permit a machine to communicate with other machines and devices, such as remotely located components, via a network. The network may provide the functionality of a local area network (LAN), a wireless network, a cloud computing environment (e.g., software as a service, platform as a service, infrastructure as a service, etc.), a client-server, a wide area network (WAN), and the like. For example, network may be a LAN or a WAN that may include other systems (including additional image processing computing systems or image-based components associated with medical imaging or radiotherapy operations).

In an example, the image processing computing system 110 may obtain image data 160 from the image data source 150, for hosting on the storage device 116 and the memory 114. In an example, the software programs operating on the image processing computing system 110 may convert medical images of one format (e.g., MRI) to another format (e.g., CT), such as by producing synthetic images, such as a pseudo-CT image. In another example, the software programs may register or associate a patient medical image (e.g., a CT image or an MR image) with that patient's dose distribution of radiotherapy treatment (e.g., also represented as an image) so that corresponding image voxels and dose voxels are appropriately associated. In yet another example, the software programs may substitute functions of the patient images such as signed distance functions or processed versions of the images that emphasize some aspect of the image information. Such functions might emphasize edges or differences in voxel textures, or other structural aspects. In another example, the software programs may visualize, hide, emphasize, or de-emphasize some aspect of anatomical features, patient measurements, patient state information, or dose or treatment information, within medical images. The storage device 116 and memory 114 may store and host data to perform these purposes, including the image data 160, patient data, and other data required to create and implement a radiation therapy treatment plan and associated patient state estimation operations.

The processing circuitry 112 may be communicatively coupled to the memory 114 and the storage device 116, and the processing circuitry 112 may be configured to execute computer executable instructions stored thereon from either the memory 114 or the storage device 116. The processing circuitry 112 may execute instructions to cause medical images from the image data 160 to be received or obtained in memory 114, and processed using the workflow logic 120. For example, the image processing computing system 110 may receive image data 160 from the image acquisition device 170 or image data sources 150 via a communication interface and network to be stored or cached in the storage device 116. The processing circuitry 112 may also send or update medical images stored in memory 114 or the storage device 116 via a communication interface to another database or data store (e.g., a medical facility database). In some examples, one or more of the systems may form a distributed computing/simulation environment that uses a network to collaboratively perform the embodiments described herein. In addition, such network may be connected to internet to communicate with servers and clients that reside remotely on the internet.

In further examples, the processing circuitry 112 may utilize software programs (e.g., a treatment planning software) along with the image data 160 and other patient data to create a radiation therapy treatment plan. In an example, the image data 160 may include 2D or 3D volumes, such as from a CT or MR. In addition, the processing circuitry 112 may utilize deep neural networks to generate an estimated patient state.

Further, such software programs may utilize workflow logic 120 to implement an IGRT path 130 or an adaptive path 135, using the techniques further discussed herein. The processing circuitry 112 may subsequently then transmit the executable radiation therapy treatment plan via a communication interface and the network to the treatment device 180, where the radiation therapy plan will be used to treat a patient with radiation via the treatment device, consistent with results of the workflow logic 130. Other outputs and uses of the software programs and the workflow logic 130 may occur with use of the image processing computing system 110.

In an example, the image data 160 may include one or more MRI images (e.g., 2D MRI, 3D MRI, 2D streaming MRI, 4D MRI, 4D volumetric MRI, 4D cine MRI, etc.), functional MRI images (e.g., fMRI, DCE-MRI, diffusion MRI), Computed Tomography (CT) images (e.g., 2D CT, Cone beam CT, 3D CT, 4D CT), ultrasound images (e.g., 2D ultrasound, 3D ultrasound, 4D ultrasound), Positron Emission Tomography (PET) images, X-ray images, fluoroscopic images, radiotherapy portal images, Single-Photo Emission Computed Tomography (SPECT) images, computer generated synthetic images (e.g., pseudo-CT images) and the like. Further, the image data 160 may also include or be associated with auxiliary information, such as segmentations/contoured images, or dose images. In an example, the image data 160 may be received from the image acquisition device 170 and stored in one or more of the image data sources 150 (e.g., a Picture Archiving and Communication System (PACS), a Vendor Neutral Archive (VNA), a medical record or information system, a data warehouse, etc.). Accordingly, the image acquisition device 170 may comprise a MRI imaging device, a CT imaging device, a PET imaging device, an ultrasound imaging device, a fluoroscopic device, a SPECT imaging device, an integrated Linear Accelerator and MRI imaging device, or other medical imaging devices for obtaining the medical images of the patient. The image data 160 may be received and stored in any type of data or any type of format (e.g., in a Digital Imaging and Communications in Medicine (DICOM) format) that the image acquisition device 170 and the image processing computing system 110 may use to perform operations consistent with the disclosed embodiments.

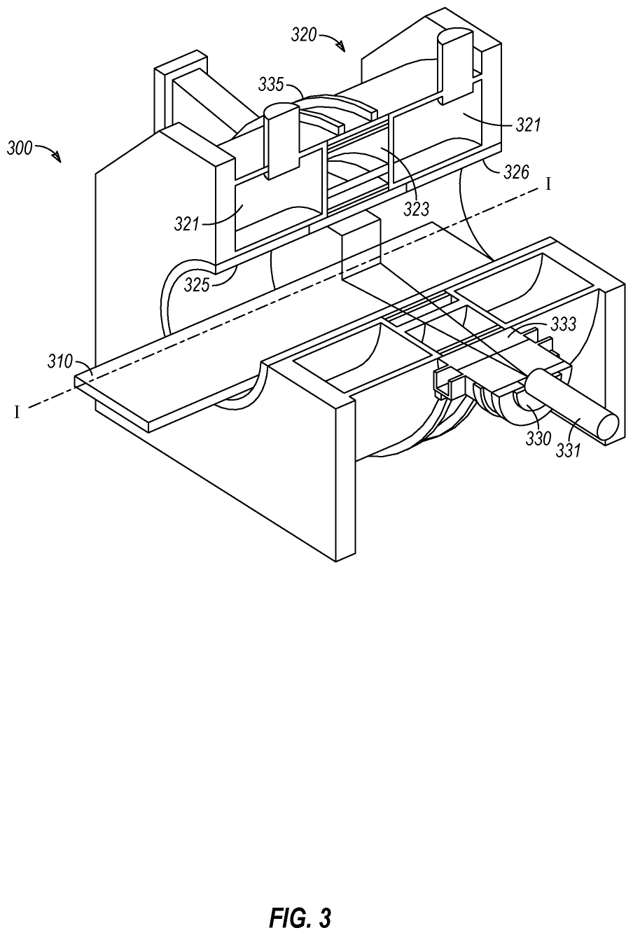

In an example, the image acquisition device 170 may be integrated with the treatment device 180 as a single apparatus (e.g., an MRI device combined with a linear accelerator, also referred to as an “MR-linac”, as shown and described in FIG. 3 below). Such an MR-linac can be used, for example, to precisely determine a location of a target organ or a target tumor in the patient, so as to direct radiation therapy accurately according to the radiation therapy treatment plan to a predetermined target. For instance, a radiation therapy treatment plan may provide information about a particular radiation dose to be applied to each patient. The radiation therapy treatment plan may also include other radiotherapy information, such as beam angles, dose-histogram-volume information, the number of radiation beams to be used during therapy, the dose per beam, and the like.

The image processing computing system 110 may communicate with an external database through a network to send/receive a plurality of various types of data related to image processing and radiotherapy operations. For example, an external database may include machine data that is information associated with the treatment device 180, the image acquisition device 170, or other machines relevant to radiotherapy or medical procedures. Machine data information may include radiation beam size, arc placement, beam on and off time duration, machine parameters, segments, multi-leaf collimator (MLC) configuration, gantry speed, MRI pulse sequence, and the like. The external database may be a storage device and may be equipped with appropriate database administration software programs. Further, such databases or data sources may include a plurality of devices or systems located either in a central or a distributed manner.

The image processing computing system 110 can collect and obtain data, and communicate with other systems, via a network using one or more communication interfaces, which are communicatively coupled to the processing circuitry 112 and the memory 114. For instance, a communication interface may provide communication connections between the image processing computing system 110 and radiotherapy system components (e.g., permitting the exchange of data with external devices). For instance, the communication interface may in some examples have appropriate interfacing circuitry from an output device 142 or an input device 144 to connect to the user interface 140, which may be a hardware keyboard, a keypad, or a touch screen through which a user may input information into the radiotherapy system.

As an example, the output device 142 may include a display device which outputs a representation of the user interface 140 and one or more aspects, visualizations, or representations of the medical images. The output device 142 may include one or more display screens that display medical images, interface information, treatment planning parameters (e.g., contours, dosages, beam angles, labels, maps, etc.) treatment plans, a target, localizing a target or tracking a target, patient state estimations (e.g., a 3D volume), or any related information to the user. The input device 144 connected to the user interface 140 may be a keyboard, a keypad, a touch screen or any type of device that a user may input information to the radiotherapy system. Alternatively, the output device 142, the input device 144, and features of the user interface 140 may be integrated into a single device such as a smartphone or tablet computer, e.g., Apple ipad®, Lenovo Thinkpad®, Samsung Galaxy®, etc.

Furthermore, any and all components of the radiotherapy system may be implemented as a virtual machine (e.g., via VMWare, Hyper-V, and the like virtualization platforms). For instance, a virtual machine can be software that functions as hardware. Therefore, a virtual machine can include at least one or more virtual processors, one or more virtual memories, and one or more virtual communication interfaces that together function as hardware. For example, the image processing computing system 110, the image data sources 150, or like components, may be implemented as a virtual machine or within a cloud-based virtualization environment.

The workflow logic 120 or other hardware or software may cause the computing system to communicate with the image data sources 150 to read images into memory 114 and the storage device 116, or store images or associated data from the memory 114 or the storage device 116 to and from the image data sources 150. For example, the image data source 150 may be configured to store and provide a plurality of images (e.g., 3D MRI, 4D MRI, 2D MRI slice images, CT images, 2D Fluoroscopy images, X-ray images, raw data from MR scans or CT scans, Digital Imaging and Communications in Medicine (DICOM) metadata, etc.) that the image data source 150 hosts, from image sets in image data 160 obtained from one or more patients via the image acquisition device 170. The image data source 150 or other databases may also store data to be used by the workflow logic 120 when executing a software program that performs operations to determine a path to follow or contour an image. Further, various databases may store machine learning models, including the network parameters constituting the model learned by the network and the resulting predicted data. The image processing computing system 110 thus may obtain and/or receive the image data 160 (e.g., 2D MRI slice images, CT images, 2D Fluoroscopy images, X-ray images, 3D MRI images, 4D MRI images, etc.) from the image data source 150, the image acquisition device 170, the treatment device 180 (e.g., a MRI-Linac), or other information systems, in connection with performing image patient state estimation as part of treatment or diagnostic operations.

The image acquisition device 170 can be configured to acquire one or more images of the patient's anatomy for a region of interest (e.g., a target organ, a target tumor or both). Each image, typically a 2D image or slice, can include one or more parameters (e.g., a 2D slice thickness, an orientation, and a location, etc.). In an example, the image acquisition device 170 can acquire a 2D slice in any orientation. For example, an orientation of the 2D slice can include a sagittal orientation, a coronal orientation, or an axial orientation. The processing circuitry 112 can adjust one or more parameters, such as the thickness and/or orientation of the 2D slice, to include the target organ and/or target tumor. In an example, 2D slices can be determined from information such as a 3D MRI volume. Such 2D slices can be acquired by the image acquisition device 170 in “real-time” while a patient is undergoing radiation therapy treatment, for example, when using the treatment device 180 (with “real-time” meaning acquiring the data in 10 milliseconds or less). In another example for some applications, real-time may include a timeframe within (e.g., up to) 200 or 300 milliseconds. In an example, real-time may include a time period fast enough for a clinical problem being solved by techniques described herein. In this example, real-time may vary depending on target speed, radiotherapy margins, lag, response time of a treatment device, etc.

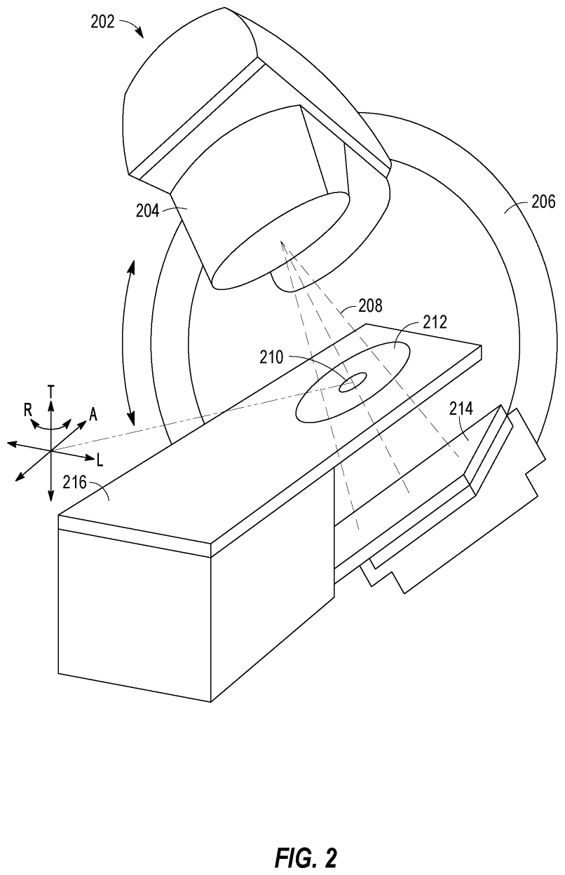

FIG. 2 illustrates an exemplary image-guided radiotherapy device 202, that includes include a radiation source, such as an X-ray source or a linear accelerator, a couch 216, an imaging detector 214, and a radiation therapy output 204. The radiation therapy device 202 may be configured to emit a radiation beam 208 to provide therapy to a patient. The radiation therapy output 204 can include one or more attenuators or collimators, such as a multi-leaf collimator (MLC).

As an example, a patient may be positioned in a region 212, supported by the treatment couch 216 to receive a radiation therapy dose according to a radiation therapy treatment plan (e.g., a treatment plan generated by the radiotherapy system of FIG. 1). The radiation therapy output 204 can be mounted or attached to a gantry 206 or other mechanical support. One or more chassis motors (not shown) may rotate the gantry 206 and the radiation therapy output 204 around couch 216 when the couch 216 is inserted into the treatment area. In an example, gantry 206 may be continuously rotatable around couch 216 when the couch 216 is inserted into the treatment area. In another example, gantry 206 may rotate to a predetermined position when the couch 216 is inserted into the treatment area. For example, the gantry 206 can be configured to rotate the therapy output 204 around an axis (“A”). Both the couch 216 and the radiation therapy output 204 can be independently moveable to other positions around the patient, such as moveable in transverse direction (“T”), moveable in a lateral direction (“L”), or as rotation about one or more other axes, such as rotation about a transverse axis (indicated as “R”). A controller communicatively connected to one or more actuators (not shown) may control the couch 216 movements or rotations in order to properly position the patient in or out of the radiation beam 208 according to a radiation therapy treatment plan. As both the couch 216 and the gantry 206 are independently moveable from one another in multiple degrees of freedom, which allows the patient to be positioned such that the radiation beam 208 precisely can target the tumor.

The coordinate system (including axes A, T, and L) shown in FIG. 2 can have an origin located at an isocenter 210. The isocenter can be defined as a location where the central axis of the radiation therapy beam 208 intersects the origin of a coordinate axis, such as to deliver a prescribed radiation dose to a location on or within a patient. Alternatively, the isocenter 210 can be defined as a location where the central axis of the radiation therapy beam 208 intersects the patient for various rotational positions of the radiation therapy output 204 as positioned by the gantry 206 around the axis A.

Gantry 206 may also have an attached imaging detector 214. The imaging detector 214 is preferably located opposite to the radiation source (output 204), and in an example, the imaging detector 214 can be located within a field of the therapy beam 208.

The imaging detector 214 can be mounted on the gantry 206 preferably opposite the radiation therapy output 204, such as to maintain alignment with the therapy beam 208. The imaging detector 214 rotating about the rotational axis as the gantry 206 rotates. In an example, the imaging detector 214 can be a flat panel detector (e.g., a direct detector or a scintillator detector). In this manner, the imaging detector 214 can be used to monitor the therapy beam 208 or the imaging detector 214 can be used for imaging the patient's anatomy, such as portal imaging. The control circuitry of radiation therapy device 202 may be integrated within the radiotherapy system or remote from it.

In an illustrative example, one or more of the couch 216, the therapy output 204, or the gantry 206 can be automatically positioned, and the therapy output 204 can establish the therapy beam 208 according to a specified dose for a particular therapy delivery instance. A sequence of therapy deliveries can be specified according to a radiation therapy treatment plan, such as using one or more different orientations or locations of the gantry 206, couch 216, or therapy output 204. The therapy deliveries can occur sequentially, but can intersect in a desired therapy locus on or within the patient, such as at the isocenter 210. A prescribed cumulative dose of radiation therapy can thereby be delivered to the therapy locus while damage to tissue nearby the therapy locus can be reduced or avoided.

Thus, FIG. 2 specifically illustrates an example of a radiation therapy device 202 operable to provide radiotherapy treatment to a patient, with a configuration where a radiation therapy output can be rotated around a central axis (e.g., an axis “A”). Other radiation therapy output configurations can be used. For example, a radiation therapy output can be mounted to a robotic arm or manipulator having multiple degrees of freedom. In yet another example, the therapy output can be fixed, such as located in a region laterally separated from the patient, and a platform supporting the patient can be used to align a radiation therapy isocenter with a specified target locus within the patient. In another example, a radiation therapy device can be a combination of a linear accelerator and an image acquisition device. In some examples, the image acquisition device may be an MRI, an X-ray, a CT, a CBCT, a spiral CT, a PET, a SPECT, an optical tomography, a fluorescence imaging, ultrasound imaging, an MR-linac, or radiotherapy portal imaging device, etc., as would be recognized by one of ordinary skill in the art.

FIG. 3 depicts an exemplary radiation therapy system 300 (e.g., known in the art as a MR-Linac) that can include combining a radiation therapy device 202 and an imaging system, such as a nuclear magnetic resonance (MR) imaging system consistent with the disclosed embodiments. As shown, system 300 may include a couch 310, an image acquisition device 320, and a radiation delivery device 330. System 300 delivers radiation therapy to a patient in accordance with a radiotherapy treatment plan. In some embodiments, image acquisition device 320 may correspond to image acquisition device 170 in FIG. 1 that may acquire images.

Couch 310 may support a patient (not shown) during a treatment session. In some implementations, couch 310 may move along a horizontal, translation axis (labelled “I”), such that couch 310 may move the patient resting on couch 310 into or out of system 300. Couch 310 may also rotate around a central vertical axis of rotation, transverse to the translation axis. To allow such movement or rotation, couch 310 may have motors (not shown) enabling the couch to move in various directions and to rotate along various axes. A controller (not shown) may control these movements or rotations in order to properly position the patient according to a treatment plan.

In some embodiments, image acquisition device 320 may include an MRI machine used to acquire 2D or 3D MRI images of the patient before, during, or after a treatment session. Image acquisition device 320 may include a magnet 321 for generating a primary magnetic field for magnetic resonance imaging. The magnetic field lines generated by operation of magnet 321 may run substantially parallel to the central translation axis I. Magnet 321 may include one or more coils with an axis that runs parallel to the translation axis I. In some embodiments, the one or more coils in magnet 321 may be spaced such that a central window 323 of magnet 321 is free of coils. In other embodiments, the coils in magnet 321 may be thin enough or of a reduced density such that they are substantially transparent to radiation of the wavelength generated by radiotherapy device 330. Image acquisition device 320 may also include one or more shielding coils, which may generate a magnetic field outside magnet 321 of approximately equal magnitude and opposite polarity in order to cancel or reduce any magnetic field outside of magnet 321. As described below, radiation source 331 of radiotherapy device 330 may be positioned in the region where the magnetic field is cancelled, at least to a first order, or reduced.

Image acquisition device 320 may also include two gradient coils 325 and 326, which may generate a gradient magnetic field that is superposed on the primary magnetic field. Coils 325 and 326 may generate a gradient in the resultant magnetic field that allows spatial encoding of the protons so that their position can be determined. Gradient coils 325 and 326 may be positioned around a common central axis with the magnet 321, and may be displaced along that central axis. The displacement may create a gap, or window, between coils 325 and 326. In the embodiments where magnet 321 also includes a central window 323 between coils, the two windows may be aligned with each other.

Image acquisition is used to track tumor movement. At times, internal or external surrogates may be used. However, implanted seeds may move from their initial positions or become dislodged during radiation therapy treatment. Also, using surrogates assumes there is a correlation between tumor motion and the displacement of the external surrogate. However, there may be phase shifts between external surrogates and tumor motion, and their positions frequently lose correlation over time. It is known that there may be mismatches between tumor and surrogates upward of 9 mm. Further, any deformation of the shape of a tumor is unknown during tracking.

An advantage of magnetic resonance imaging (MRI) is in the superior soft tissue contrast that is provided to visualize the tumor in more detail. Using a plurality of intra-fractional MR images allows the determination of both shape and position (e.g., centroid) of a tumor. In addition, MRI images improve any manual contouring performed by, for example, a radiation oncologist, even when auto-contouring software (e.g., ABAS®) is utilized. This is because of the high contrast between the tumor target and the background region provided by MR images.

Another advantage of using an MR-Linac system is that a treatment beam can be continuously on and thereby executing intra-fractional tracking of the target tumor. For instance, optical tracking devices or stereoscopic x-ray fluoroscopy systems can detect tumor position at 30 Hz by using tumor surrogates. With MRI, the imaging acquisition rates are faster (e.g., 3-6 fps). Therefore, the centroid position of the target may be determined, artificial intelligence (e.g., neural network) software can predict a future target position. An added advantage of intra-fractional tracking by using an MR-Linac is that the by being able to predict a future target location, the leaves of the multi-leaf collimator (MLC) will be able to conform to the target contour a its predicted future position. Thus, predicting future tumor position using MRI occurs at the same rate as imaging frequency during tracking. By being able to track the movement of a target tumor clearly using detailed MRI imaging allows for the delivery of a highly conformal radiation dose to the moving target.

In some embodiments, image acquisition device 320 may be an imaging device other than an MRI, such as an X-ray, a CT, a CBCT, a spiral CT, a PET, a SPECT, an optical tomography, a fluorescence imaging, ultrasound imaging, or radiotherapy portal imaging device, etc. As would be recognized by one of ordinary skill in the art, the above description of image acquisition device 320 concerns certain embodiments and is not intended to be limiting.

Radiotherapy device 330 may include the source of radiation 331, such as an X-ray source or a linear accelerator, and a multi-leaf collimator (MLC) 333. Radiotherapy device 330 may be mounted on a chassis 335. One or more chassis motors (not shown) may rotate chassis 335 around couch 310 when couch 310 is inserted into the treatment area. In an embodiment, chassis 335 may be continuously rotatable around couch 310, when couch 310 is inserted into the treatment area. Chassis 335 may also have an attached radiation detector (not shown), preferably located opposite to radiation source 331 and with the rotational axis of chassis 335 positioned between radiation source 331 and the detector. Further, device 330 may include control circuitry (not shown) used to control, for example, one or more of couch 310, image acquisition device 320, and radiotherapy device 330. The control circuitry of radiotherapy device 330 may be integrated within system 300 or remote from it.

During a radiotherapy treatment session, a patient may be positioned on couch 310. System 300 may then move couch 310 into the treatment area defined by magnetic coils 321, 325, 326, and chassis 335. Control circuitry may then control radiation source 331, MLC 333, and the chassis motor(s) to deliver radiation to the patient through the window between coils 325 and 326 according to a radiotherapy treatment plan.

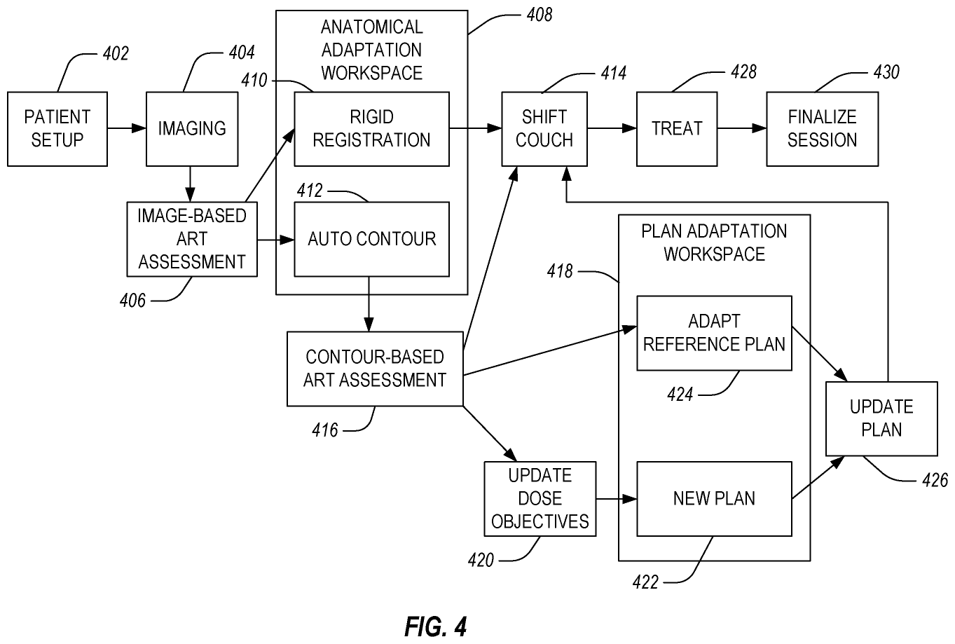

FIG. 4 illustrates an exemplary flow diagram for planning radiotherapy treatment using an IGRT and adaptive radiotherapy workflow. The flow diagram shows an IGRT workflow modified to include optional adaptive radiotherapy workflow elements. A path through operations 402, 404, 410, 414, 428, and 430 represents a typical IGRT workflow. The workflow of FIG. 4 includes optional adaptive operations.

The workflow includes patient setup 402, and imaging 404, which may include CBCT, QCBCT, etc. After imaging 404, instead of proceeding to rigid registration 410, the adaptive workflow includes performing an image-based ART assessment operation 406. An image captured in the imaging operation 404 may be used to assess whether to return to the IGRT workflow by performing rigid registration 410, or to continue with an adaptive workflow by automatically contouring the image at operation 412. The image-based ART assessment 406 is done without contours. This saves time and resources by not automatically contouring in examples where rigid registration 410 is appropriate. The image-based ART assessment 406 may include using an AI-based plan prediction to calculate an expected dose or volume metric on the image from imaging 404. The image-based ART assessment 406 automatically estimating an IGRT shift and calculating dose or volume metrics. The metrics may be compared to corresponding thresholds to determine what to do next in anatomical adaptation workspace 408. In an example, an AI based-classifier may be trained to determine whether to continue with IGRT or move to further ART workflow operations, for example based on population image data. In an example, a rigid registration in a region of interest around the target is first performed after the CBCT acquisition, to align it to the planning CT such that the targets overlap. This may include shifts or rotations. Then the image-based ART assessment may be carried out based on the shifted CBCT image. For example, changing the order of the image-based ART assessment 406 and the rigid registration 410.

When the image-based ART assessment 406 results in a determination to continue with the IGRT workflow, at the anatomical adaptation workspace 408, rigid registration 410 may be performed, and the IGRT workflow may continue with shifting the couch 414, etc. When the image-based ART assessment 406 results in a determination to continue with the adaptive workflow, the anatomical adaptation workspace 408 may include performing an auto contour operation 412 on the image from the imaging operation 404.

A contour-based ART assessment may be performed at operation 416 after the auto contouring 412. The auto contour may be used as an input (the operation 416 may optionally use the image as well as the contour as input). The contour-based ART assessment operation 41y6 may output a determination of whether to continue with an IGRT workflow or proceed with an adaptive workflow. The output may include a confidence or probability score, in some examples. The output may indicate whether to adapt a reference plan (e.g., a warm start) at operation 424, or to generate a new plan by updating dose objectives 420 and generating a new plan 422. For example, a plan adaptation workspace 418 may be used to adapt or generate a new plan. The contour-based ART assessment 416 may perform an automatic calculation of one or more new potential dose objectives that may be achieved. The one or more dose objectives may be compared to an IGRT dose metrics or to original plan metrics. When the new one or more dose objectives are not better (or only marginally better) than IGRT metrics, the workflow may revert to the IGRT workflow by shifting the couch at 414. When the new one or more dose objectives are better than the IGRT objectives, but are not better (or only marginally better) than the original plan metrics, then the plan adaptation workspace 418 may use the adapt reference plan operation 424 to adapt the previously generated plan. When the new one or more dose objectives are better or significantly better (e.g., exceed a threshold improvement to treatment) than the original plan metrics, then the plan adaptation workspace 418 may use the new plan 422 operation after the new one or more dose objectives are generated at operation 420. The new plan 422 may re-optimize the original plan from scratch using the new one or more dose objectives. After the plan is updated, it is output at operation 426, used to shift the couch at operation 414, and proceed with treatment 428, and finalizing the session at operation 430.

The thresholds described above may be dependent on a protocol that decides whether specialized clinical staff is needed. For example, one clinic may choose that any minor improvements should go to adaptation, whereas another may choose that only very significant improvements should go to adaptation, in order to avoid requiring a physician to approve.

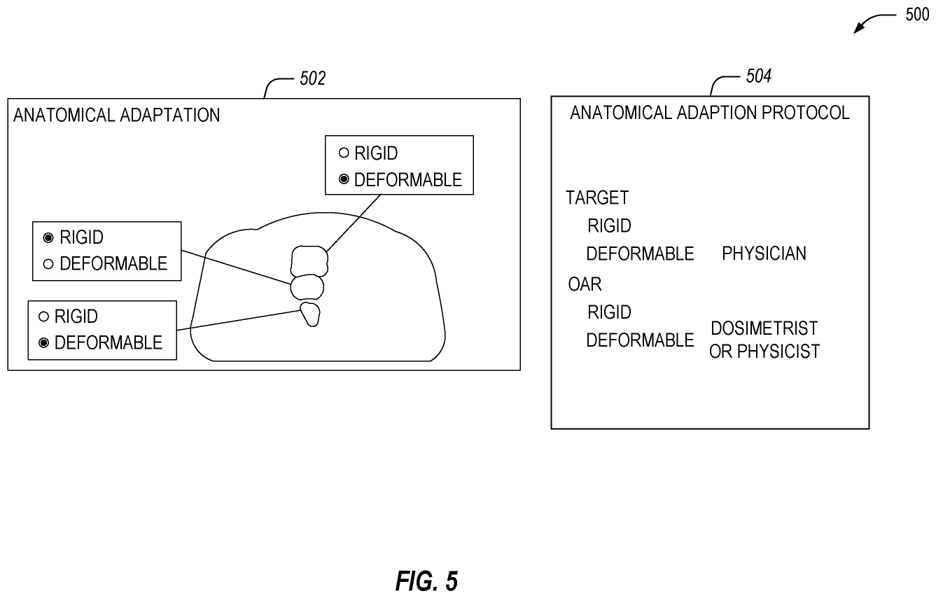

FIG. 5 illustrates an exemplary user interface 500 showing example rigid and deformable structures. The user interface 500 includes an anatomical adaptation component 502 and an anatomical adaptation protocol component 504. The anatomical adaptation component 502 may use one or more guide structures, identified in the user interface 500 as being rigid or deformable. These guide structures may include initial structures used to assist in the image registration process, and may not be the actual structures that are used for adaptation. In an example, the guide structures are quickly generated with an autocontouring technique, and may be edited by any staff if preferred. Each guide structure may be independently toggled between a ‘rigid’ or ‘deformable’ state. Deformable guide structures may be used to guide or constrain the deformable image registration between the planning and treatment images. The resulting deformation vector field (DVF) may be used to propagate the planning structures to the new image. Rigid guide structures may be used to guide a rigid registration between the planning and treatment images, for example using only voxels within the structure plus a margin. The planning structure may be shifted or rotated to the new location by the resulting registration.

Using the guide structure concept, the deformable registration may be improved, or the complexity level of the contour generation may be tied to approvals required by specific specialized staff. For example, no approval may be required if all guide structures are rigid. In some centers, a physician may not be required for deformable OARs, although they may require a dosimetrist or physicist. When the target itself is deformable, a physician approval may be required. Deformable target propagation may be handled with care. Tumors may shrink over the course of treatment, and it may not be safe to adapt to this shrinking tissue since the original volume may still contain microscopic disease. Surrounding tissues may move inward with the regressing tumor or remain in its initial location while the tissue regresses. Similar considerations may be made regarding deformable propagation of margins. Outside of a clinical trial, it may be useful to rigidly propagate the target structures than to deform them. Allowing the target to be individually selected as rigid may, in some centers, allow the adaptive treatment to progress without physician approval even though the OARs are deformable.

The anatomical adaptation protocol component 504 here may be clinic-, anatomy-, or physician-dependent. This may be determined prior to a first fraction. The anatomical adaptation component 502 may include one or more thresholds on deformations, such as specialized staff is only called in if structures are deformed beyond a certain threshold.

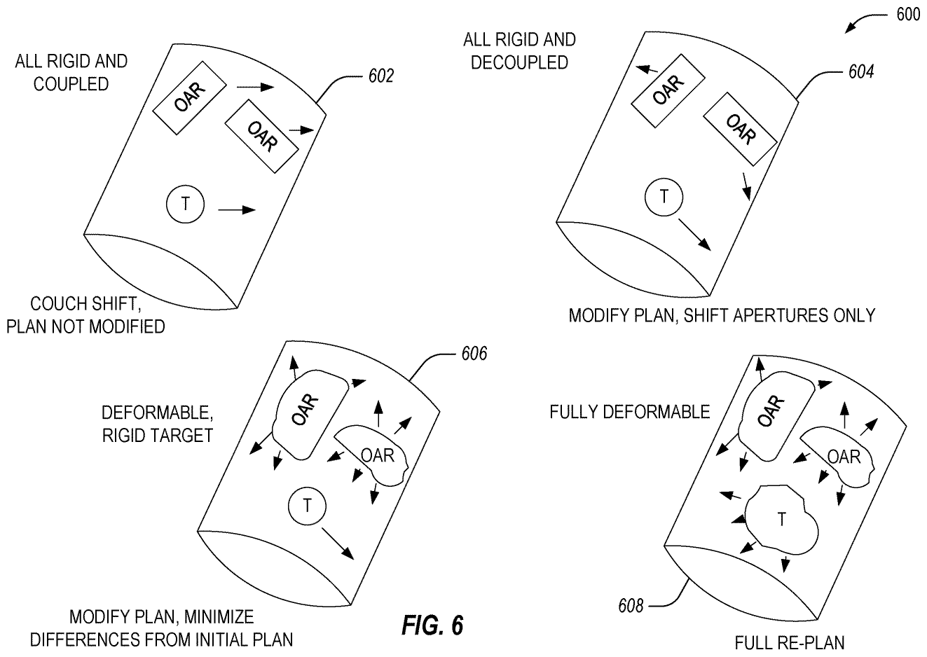

FIG. 6 illustrates an exemplary diagram 600 showing adaptive radiotherapy configurations. The diagram 600 includes a set of example adaptation states 602, 604, 606, and 608. These example adaptation states may represent points on a range of possible example adaptation states for example from having all organs at risk (OAR) and a target rigid and coupled (and potentially not moved at all), as in state 602 to having fully deformable OARs and target, as in state 608. In between the two ends of the range (which may be states 602 and 608 or may be otherwise) include states 604 and 606. State 604 includes a rigid but decoupled OAR and state 606 includes deformable OARs, but a rigid target.

Online adaptive radiotherapy (ART) enables the generation of a new treatment plan for each treatment fraction based on online images with the patient in setup position. The whole treatment planning process is compressed to fit within a treatment session. Processes that traditionally take days or even weeks are compressed into minutes. To achieve this, manual processes may be automated. Unlike non-adaptive radiotherapy treatments, physician presence is generally required for each adaptive treatment session to edit and validate these automatic processes. This necessity for physician presence limits the mainstream adoption of online adaptive radiotherapy. The systems and techniques described herein provide a workflow that integrates adaptive and non-adaptive workflows and limits physician presence to only when needed.

The states shown in FIG. 6 may correspond to anatomical adaptation or plan adaptation, for example to separately determine whether physician presence is needed. Anatomical adaptation may include calculating a rigid registration between a planning and session image, for example assuming that the targets and OARs are coupled and all move in tandem, as represented in state 602. This is the assumption commonly made in IGRT workflows and generally does not necessitate physician presence. Rigid registration may include translations or rotations, in some examples. For some treatment sites, such as prostate cancer, rotations may be a significant component of motion.

A further level of complexity, represented in state 604, may include identifying that the target and OARs move rigidly, but independently to one another. This adds more flexibility towards adaptive functionality and is likely to not require physician presence at most institutions for anatomical adaptation.

A further level of complexity, shown in state 606, may be used with ART, since estate 606 includes a deformable image registration for the entire patient anatomy, except the target which remains rigid. Dosimetrists may generally be proficient in contouring normal anatomy, and some centers may be willing to allow a dosimetrist to contour the OARs in state 606 without physician approval or direct supervision (a QA by the physician may occur). State 606 may be more likely to allow a dosimetrist to perform the contouring when imaging included high quality CBCT or synthetic CTs (sCTs), which resemble the planning CT closely. The similarity of CBCT or sCT to the planning CT allows a user to observe relative changes between images rather than having to identify structures from scratch.

Modifying the target volume (T) may be controversial and may lead to errors if not considered carefully since it may include microscopic disease, which does not necessarily deform in the same manner as the gross target anatomy. Some clinicians recommend rigid propagation of the target outside of a clinical trial, except for cases which are defined by organ boundaries such as prostate or cervix. Physician presence may not be necessary, at least for some centers, for workflows based on state 606. It is relatively common practice for OARs to be contoured by dosimetrists, and rigid target volume shifts are commonly implemented without physician approval in IGRT sessions.

State 608 includes deformation of the target volume, which is likely to require physician presence in most if not all institutions. In some examples, such as for small-to-medium deformations on high quality images with targets defined by organ boundaries, some centers may relax the requirement to have a physician present even in state 608, such as after a user or clinic has experience with online ART. Some clinicians recommend that under the context of clinical target volume (CTV) deformations, the PTV is to be either rigidly translated or margins re-expanded from the deformed CTV.

Regarding plan adaptation, state 602 represents shifting or rotating the couch. This is a limited form of plan adaptation since the dose distribution is affected by the shifting or rotation of the couch. IGRT is typically performed for state 602, but in some examples the couch shifts may be calculated based on any of anatomical plan adaptations described above.

State 604 represents plan adaptation that allows changing the MLC leaf positions in a limited form. The aperture shapes may be kept constant, but allowed to shift or rotate relative to the patient. The weight of each beam may be modified to compensate for dose differences caused by the shifts.

State 606 represents plan adaptation that attempts to reproduce the initial treatment plan as closely as possible, while adapting to the new anatomy. This is sometimes referred to as a ‘warm start’ optimization. For example, segment aperture morphing (SAM) techniques may be used, the optimization may penalize deviations from the initial treatment plan, the goal can be to match the deformed initial dose distribution rather than the original treatment objectives, or the like.

State 608 represents full re-plan of the patient based on the adapted anatomy, which may be particularly useful for large anatomical changes or to attempt to improve on the original treatment plan if the adapted anatomy allows.

The anatomical adaptation and plan adaptation described for FIG. 6 are examples, and choices are not limited to these descriptions. By allowing a user to escalate independently for anatomical and plan adaptations, a trade-off between institution and regional requirements for physician presence may be balanced with clinical value. The workflow described herein may allow a clinician to identify an appropriate balance on a session-by-session basis to significantly lower the threshold for adoption of online ART.

For many cases, IGRT, or a compromise between IGRT and full adaptive, may be sufficient. The systems and techniques described herein allow a user to adapt a workflow to independent custom levels of anatomical or plan adaptations that balance clinical benefit and institution specific physician presence requirements. For example, during a treatment course many days of treatment are typically required. On the first day of treatment, a full adaptation may be needed to compensate for large anatomical changes requiring physician presence at the treatment machine. On the second day of treatment, the changes may be minimal and a simple IGRT shift of the couch may be sufficient, requiring no physician presence at the treatment machine. Each treatment instance may require an escalation assessment to determine whether physician presence is required based on institution-specific guidelines.

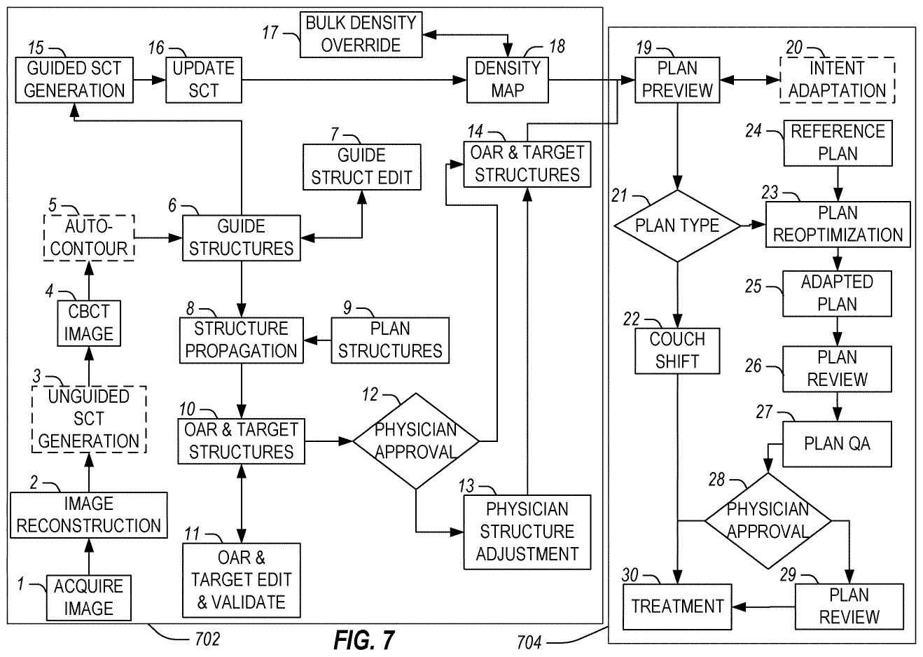

FIG. 7 illustrates an exemplary block diagram for an adaptive radiotherapy workflow. The anatomic adaptation 702 and plan adaptation 704 blocks are separately boxed off, but either or both may be referred to as a workflow. After initial patient set-up, a kV image acquisition block 1 and CBCT image reconstruction block 2 are initiated, which may include initiating them at least partially in parallel. An unguided sCT algorithm is optionally generated block 3 and stored along with the CBCT image block 4. Although it is not explicitly shown, registered MR and PET images from the planning stage may be added to the CBCT image block 4. The sCT is referred to here as unguided since there are no structures to guide or constrain the algorithm. The goal of this sCT is to improve image quality and make the image more consistent with diagnostic CT. Errors in this initial sCT generation are tolerable as will be discussed later in the workflow. An AI implementation of sCT generation may be used at block 3, since DVF implementations are more robust when guided by structures (as will be discussed below, a DVF based sCT approach can be used in guided SCT generation block 15).

In auto-contour block 5 an optional autocontouring process is initiated to generate guide structures and the result is stored in block 6. Guide structures may include a set of structures that are be used to guide downstream rigid or deformable image registration algorithms. In block 7, a user may edit the guide structures stored at block 6, or toggle their status as either rigid guides (e.g., translations only or translations and rotations), or deformable guides. In the rigid case, the planning structure shapes may not change in the adaptation session, but may shift or rotate. This case may obviate the need for physician presence. Using all rigid guides, or deformable guides for OARs and rigid guides for targets, may satisfy this need in some centers. A physician approval status may be displayed (e.g., on a user interface) as a user moves through the workflow. For example, when the user switches from a rigid to a deformable structure guide in block 7, the status may switch from no physician needed to yes physician needed. The logic used to evaluate this status may be institution or center specific.

Block 8 shows a structure propagation step, registering the CBCT (or sCT) image to a planning CT and using these registrations to propagate the initial plan structures (block 9) to the current patient anatomy. For rigid guides, the rigid registration may be performed in a region of interest (ROI) defined by the guide (e.g., with an expansion around the guide structure). For deformable guides they may be defined by a deformable registration that is guided or constrained by the guides. The resulting OAR and target structures may be stored in block 10, and may be edited or validated by a user (e.g., a non-physician).

When guide structures are not autocontoured in optional step 5, they may be manually contoured. The manual contouring may be reserved for cases where only rigid registrations are performed, in which case step 6 can include setting up regions of interest for rigid registration around target and OARs.

Block 12 may branch based on a current physician approval status. When the status is no, the OAR and target structures may be stored directly in block 14, and forwarded to plan adaptation along with the density map of block 17. When the status is yes, for example when at least one deformable guide was selected in block 7, then the physician may be provided an opportunity to tweak structures and approve the structures before moving forward to plan adaptation. Note that while waiting for the physician in block 13, the plan adaptation may continue, with the proviso that it may need to be revised if the physician edits the contours.

Block 15 is a guided sCT generation step. The goal is to use the guide structures to improve upon the unguided sCT generation of block 3 for purposes of creating a density map. The updated sCT is stored in block 16 and the corresponding density map stored in block 17. The density map may be manually over-ridden by assigning bulk densities to structures in block 18. At the end of the anatomical adaptation section of the workflow, the structures and density map required for treatment planning are available for plan adaptation. Although automatic processes such as autocontouring, image registration and sCT generation have been used, failures may be mitigated (e.g., at the expense of time) via manual contour editing and bulk density over-ride steps.

Block 19 is a plan preview step where an estimation of different plan types is shown to guide the user in decisions related to subsequent plan re-optimization. For example, the user may be shown dose distributions and metrics for each of the blocks of FIG. 4. This may be performed using an AI-based plan prediction component. Optionally, the user may decide to modify the intent at block 20 based upon the plan preview. The status of the physician approval flag may change depending on the plan type selected or when the intent is modified, such as according to site-specific logic.

After selecting the plan type at block 21, the workflow may branch to either a couch shift or rotation at block 22, or to plan re-optimization at block 23. Plan re-optimization generates an adapted plan at block 25, for example according to the plan type selection. The reference plan of block 24 may be used as input depending on the plan type.

A first plan review may be performed by a non-physician at block 26, followed by Plan QA at block 27. When the physician approval status is no, the workflow may proceed to treatment. When it is yes, a second physician plan review and approval may be performed at block 29. The last block 30 is treatment, for example according to the plan.

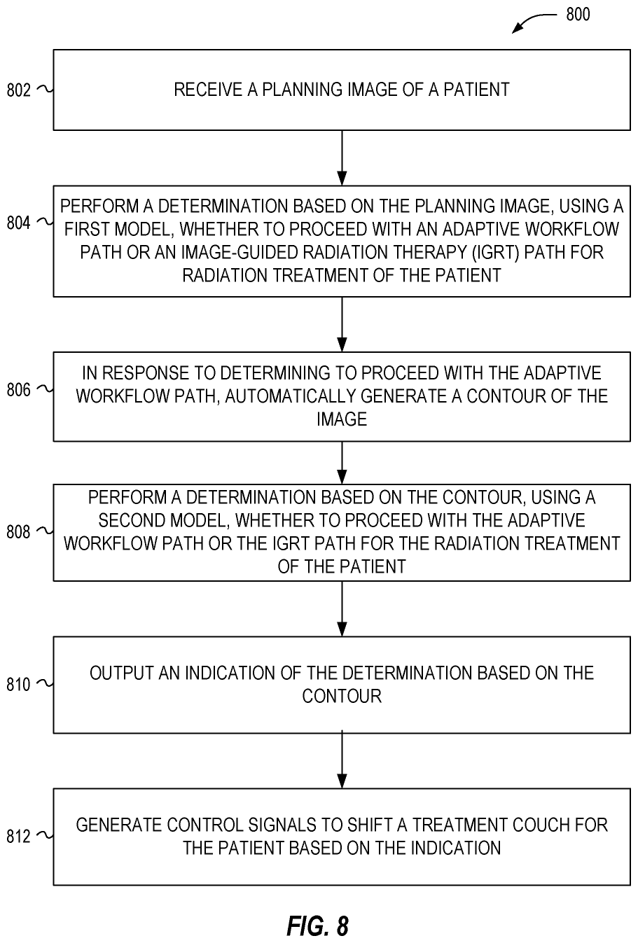

FIG. 8 illustrates a flowchart 800 of exemplary operations for planning radiotherapy treatment using an IGRT and adaptive radiotherapy workflow. The flowchart 800 includes an operation 802 to receive a planning image of a patient. In an example, the planning image is a cone-beam computed tomography (CBCT) image.

The flowchart 800 includes an operation 804 to perform a determination based on the planning image, using a first model, whether to proceed with an adaptive workflow path or an image-guided radiation therapy (IGRT) path for radiation treatment of the patient. Operation 804 may include determining, using the first mode, dose and volume metrics for radiation treatment of the patient based on the image and comparing the dose and volume metrics to a threshold.

The flowchart 800 includes an operation 806 to in response to determining to proceed with the adaptive workflow path, automatically generate a contour of the image. Operation 806 may include automatically generating a contour of a first set of rigid structures of the image and a second set of deformable structures of the image.

The flowchart 800 includes an operation 808 to perform a determination based on the contour, using a second model, whether to proceed with the adaptive workflow path or the IGRT path for the radiation treatment of the patient. In some examples, the first model or the second model is a trained machine learning model including a convolutional neural network (CNN). In other examples, the first model or the second model is a trained classifier. Operation 808 may include in response to the determination based on the contour indicating to proceed with the adaptive workflow path, further determining whether to generate a new plan or to update a reference plan. This example may include in response to determining to generate the new plan, updating dose objectives based on the contour. In an example, operation 808 may include determining a level of adaptation, such as one or more of: shifting the apertures of the reference plan, reweighting apertures of the reference plan, warping the apertures of the reference plan, re-optimizing the fluency of the reference plan, updating the objectives of the reference plan, generating a new plan that minimizes differences between the new plan and the reference plan, generating a new plan without consideration of the reference plan, or the like.

The flowchart 800 includes an operation 810 to output an indication of the determination based on the contour. In some examples, operation 810 may include in response to the determination based on the contour indicating to proceed with the IGRT path, outputting the contour. In these examples, generating the control signals may include using a reference plan to shift the treatment couch. In an example, operation 810 includes in response to the determination based on the planning image indicating to proceed with the IGRT path, outputting an indication on a physician user interface to perform rigid registration on the image. The flowchart 800 includes an operation 812 to generate control signals to shift a treatment couch for the patient based on the indication. In some examples, outputting the indication may include outputting a level or percentage related to the determination. For example, a confidence percentage for whether to proceed with an IGRT or an adaptive workflow may be provided.

The above detailed description includes references to the accompanying drawings, which form a part of the detailed description. The drawings show, by way of illustration but not by way of limitation, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a,” “an,” “the,” and “said” are used when introducing elements of aspects of the invention or in the embodiments thereof, as is common in patent documents, to include one or more than one or more of the elements, independent of any other instances or usages of “at least one” or “one or more.” In this document, the term “or” is used to refer to a nonexclusive or, such that “A or B” includes “A but not B,” “B but not A,” and “A and B,” unless otherwise indicated.

In the appended claims, the terms “including” and “in which” are used as the plain-English equivalents of the respective terms “comprising” and “wherein.” Also, in the following claims, the terms “comprising,” “including,” and “having” are intended to be open-ended to mean that there may be additional elements other than the listed elements, such that after such a term (e.g., comprising, including, having) in a claim are still deemed to fall within the scope of that claim. Moreover, in the following claims, the terms “first,” “second,” and “third,” etc., are used merely as labels, and are not intended to impose numerical requirements on their objects.

The present invention also relates to a computing system adapted, configured, or operated for performing the operations herein. This system may be specially constructed for the required purposes, or it may comprise a general purpose computer selectively activated or reconfigured by a computer program (e.g., instructions, code, etc.) stored in the computer. The order of execution or performance of the operations in embodiments of the invention illustrated and described herein is not essential, unless otherwise specified. That is, the operations may be performed in any order, unless otherwise specified, and embodiments of the invention may include additional or fewer operations than those disclosed herein. For example, it is contemplated that executing or performing a particular operation before, contemporaneously with, or after another operation is within the scope of aspects of the invention.

In view of the above, it will be seen that the several objects of the invention are achieved and other advantageous results attained. Having described aspects of the invention in detail, it will be apparent that modifications and variations are possible without departing from the scope of aspects of the invention as defined in the appended claims. As various changes could be made in the above constructions, products, and methods without departing from the scope of aspects of the invention, it is intended that all matter contained in the above description and shown in the accompanying drawings shall be interpreted as illustrative and not in a limiting sense.

The above description is intended to be illustrative, and not restrictive. For example, the above-described examples (or one or more aspects thereof) may be used in combination with each other. In addition, many modifications may be made to adapt a particular situation or material to the teachings of the invention without departing from its scope. While the dimensions, types of materials and example parameters, functions, and implementations described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled.

Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

Example 1 is a method comprising: receiving a planning image of a patient; performing a determination based on the planning image, using a first model, whether to proceed with an adaptive workflow path or an image-guided radiation therapy (IGRT) path for radiation treatment of the patient; in response to determining to proceed with the adaptive workflow path, automatically generating a contour of the image; performing a determination based on the contour, using a second model, whether to proceed with the adaptive workflow path or the IGRT path for the radiation treatment of the patient; outputting an indication of the determination based on the contour; and generating control signals to shift a treatment couch for the patient based on the indication.

In Example 2, the subject matter of Example 1 includes, wherein the planning image is a cone-beam computed tomography (CBCT) image.

In Example 3, the subject matter of Examples 1-2 includes, wherein the first model or the second model is a trained machine learning model including a convolutional neural network (CNN).

In Example 4, the subject matter of Examples 1-3 includes, wherein the first model or the second model is a trained classifier.

In Example 5, the subject matter of Examples 1-4 includes, wherein automatic generating the contour of the image includes automatically generating a contour of a first set of rigid structures of the image and a second set of deformable structures of the image.

In Example 6, the subject matter of Examples 1-5 includes, wherein, in response to the determination based on the contour indicating to proceed with the adaptive workflow path, further determining whether to generate a new plan or to update a reference plan.

In Example 7, the subject matter of Example 6 includes, wherein in response to determining to generate the new plan, updating dose objectives based on the contour.

In Example 8, the subject matter of Examples 1-7 includes, wherein in response to the determination based on the contour indicating to proceed with the IGRT path, outputting the contour and wherein generating the control signals includes using a reference plan to shift the treatment couch.

In Example 9, the subject matter of Examples 1-8 includes, wherein in response to the determination based on the planning image indicating to proceed with the IGRT path, outputting an indication on a physician user interface to perform rigid registration on the image.

In Example 10, the subject matter of Examples 1-9 includes, wherein performing the determination based on the planning image includes: determining, using the first mode, dose and volume metrics for radiation treatment of the patient based on the image; and comparing the dose and volume metrics to a threshold.

Example 11 is at least one machine-readable medium including instructions, which when executed by processing circuitry, cause the processing circuitry to perform operations to: receive a planning image of a patient; perform a determination based on the planning image, using a first model, whether to proceed with an adaptive workflow path or an image-guided radiation therapy (IGRT) path for radiation treatment of the patient; automatically generate in response to determining to proceed with the adaptive workflow path, a contour of the image; perform a determination based on the contour, using a second model, whether to proceed with the adaptive workflow path or the IGRT path for the radiation treatment of the patient; output an indication of the determination based on the contour; and generate control signals to shift a treatment couch for the patient based on the indication.

In Example 12, the subject matter of Example 11 includes, wherein the planning image is a cone-beam computed tomography (CBCT) image.

In Example 13, the subject matter of Examples 11-12 includes, wherein the first model or the second model is a trained machine learning model including a convolutional neural network (CNN).

In Example 14, the subject matter of Examples 11-13 includes, wherein the first model or the second model is a trained classifier.

In Example 15, the subject matter of Examples 11-14 includes, wherein automatic generation of the contour of the image includes automatically generation of a contour of a first set of rigid structures of the image and a second set of deformable structures of the image.

In Example 16, the subject matter of Examples 11-15 includes, wherein, in response to the determination based on the contour indicating to proceed with the adaptive workflow path, the instructions include a further determination of whether to generate a new plan or to update a reference plan.

In Example 17, the subject matter of Example 16 includes, wherein in response to determining to generate the new plan, the instructions include an update of dose objectives based on the contour.

In Example 18, the subject matter of Examples 11-17 includes, wherein in response to the determination based on the contour indicating to proceed with the IGRT path, the instructions further cause the processing circuitry to output the contour and wherein generating the control signals includes using a reference plan to shift the treatment couch.

In Example 19, the subject matter of Examples 11-18 includes, wherein in response to the determination based on the planning image indicating to proceed with the IGRT path, the instructions further cause the processing circuitry to output an indication on a physician user interface to perform rigid registration on the image.

In Example 20, the subject matter of Examples 11-19 includes, wherein the instructions to perform the determination based on the planning image include to: determine, using the first mode, dose and volume metrics for radiation treatment of the patient based on the image; and compare the dose and volume metrics to a threshold.

Example 21 is a method comprising: receiving a planning image of a patient; performing a determination based on the planning image, using a first model, whether to proceed with an adaptive workflow path or an image-guided radiation therapy (IGRT) path for radiation treatment of the patient; in response to determining to proceed with the IGRT path, outputting an indication to perform rigid registration; and receiving a user input contour of the planning image; and generating control signals to shift a treatment couch for the patient based on the user input contour.

Example 22 is a method comprising: receiving a planning image of a patient; performing a determination based on the planning image, using a first model, whether to proceed with an adaptive workflow path or an image-guided radiation therapy (IGRT) path for radiation treatment of the patient; in response to determining to proceed with the adaptive workflow path, automatically generating a contour of the image; performing a determination based on the contour, using a second model, whether to proceed with the adaptive workflow path or the IGRT path for the radiation treatment of the patient; in response to determining to proceed with the adaptive workflow path, adapting a reference treatment plan; and generating control signals to shift a treatment couch for the patient based on the adapted reference plan.

Example 23 is a method comprising: receiving a planning image of a patient; performing a determination based on the planning image, using a first model, whether to proceed with an adaptive workflow path or an image-guided radiation therapy (IGRT) path for radiation treatment of the patient; in response to determining to proceed with the adaptive workflow path, automatically generating a contour of the image; performing a determination based on the contour, using a second model, whether to proceed with the adaptive workflow path or the IGRT path for the radiation treatment of the patient; in response to determining to proceed with the adaptive workflow path, updating dose objectives; generating a new treatment plan based on the updated dose objectives; and generating control signals to shift a treatment couch for the patient based on the new treatment plan.

Example 24 is at least one machine-readable medium including instructions that, when executed by processing circuitry, cause the processing circuitry to perform operations to implement of any of Examples 1-23.

Example 25 is an apparatus comprising means to implement of any of Examples 1-23.

Example 26 is a system to implement of any of Examples 1-23.

Example 27 is a method to implement of any of Examples 1-23.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

1. A method comprising:
receiving a planning image of a patient;
performing, using processing circuitry, a determination based on the planning image, using a first model, whether to proceed with an adaptive workflow path or an image-guided radiation therapy (IGRT) path for radiation treatment of the patient;
in response to determining to proceed with the adaptive workflow path, automatically generating a contour of the image;
performing, using the processing circuitry, a determination based on the contour, using a second model, whether to proceed with the adaptive workflow path or the IGRT path for the radiation treatment of the patient;
outputting an indication of the determination based on the contour; and
generating control signals to shift a treatment couch for the patient based on the indication.

2. The method of claim 1, wherein the planning image is a cone-beam computed tomography (CBCT) image.

3. The method of claim 1, wherein the first model or the second model is a trained machine learning model including a convolutional neural network (CNN).

4. The method of claim 1, wherein the first model or the second model is a trained classifier.

5. The method of claim 1, wherein automatically generating the contour of the image includes automatically generating a contour of a first set of rigid structures of the image and a second set of deformable structures of the image.

6. The method of claim 1, wherein, in response to the determination based on the contour indicating to proceed with the adaptive workflow path, further determining whether to generate a new plan or to update a reference plan.

7. The method of claim 6, wherein in response to determining to generate the new plan, updating dose objectives based on the contour.

8. The method of claim 1, wherein in response to the determination based on the contour indicating to proceed with the IGRT path, outputting the contour for display and wherein generating the control signals includes using a reference plan to shift the treatment couch.

9. The method of claim 1, wherein in response to the determination based on the planning image indicating to proceed with the IGRT path, outputting an indication on a physician user interface to perform rigid registration on the image.

10. The method of claim 1, wherein performing the determination based on the planning image includes:
determining, using the first model, dose and volume metrics for radiation treatment of the patient based on the image; and
comparing the dose and volume metrics to a threshold.

11. At least one machine-readable medium including instructions, which when executed by processing circuitry, cause the processing circuitry to perform operations to:
receive a planning image of a patient;
perform a determination based on the planning image, using a first model, whether to proceed with an adaptive workflow path or an image-guided radiation therapy (IGRT) path for radiation treatment of the patient;
automatically generate in response to determining to proceed with the adaptive workflow path, a contour of the image;
perform a determination based on the contour, using a second model, whether to proceed with the adaptive workflow path or the IGRT path for the radiation treatment of the patient;
output an indication of the determination based on the contour; and
generate control signals to shift a treatment couch for the patient based on the indication.

12. The at least one machine-readable medium of claim 11, wherein the planning image is a cone-beam computed tomography (CBCT) image.

13. The at least one machine-readable medium of claim 11, wherein the first model or the second model is a trained machine learning model including a convolutional neural network (CNN).

14. The at least one machine-readable medium of claim 11, wherein the first model or the second model is a trained classifier.

15. The at least one machine-readable medium of claim 11, wherein automatic generation of the contour of the image includes automatically generation of a contour of a first set of rigid structures of the image and a second set of deformable structures of the image.

16. The at least one machine-readable medium of claim 11, wherein, in response to the determination based on the contour indicating to proceed with the adaptive workflow path, the instructions include a further determination of whether to generate a new plan or to update a reference plan.

17. The at least one machine-readable medium of claim 16, wherein in response to determining to generate the new plan, the instructions include an update of dose objectives based on the contour.

18. The at least one machine-readable medium of claim 11, wherein in response to the determination based on the contour indicating to proceed with the IGRT path, the instructions further cause the processing circuitry to output the contour and wherein generating the control signals includes using a reference plan to shift the treatment couch.

19. The at least one machine-readable medium of claim 11, wherein in response to the determination based on the planning image indicating to proceed with the IGRT path, the instructions further cause the processing circuitry to output an indication on a physician user interface to perform rigid registration on the image.

20. The at least one machine-readable medium of claim 11, of wherein the instructions to perform the determination based on the planning image include to:
determine, using the first model, dose and volume metrics for radiation treatment of the patient based on the image; and
compare the dose and volume metrics to a threshold.

21-23. (canceled)
